(19) United States
(12) Patent Application Publication
(10) Pub. No.: US 2020/0197128 A1
(43) Pub. Date: Jun. 25, 2020

(54) JAW SUPPORT

(71) Applicant: FREEDOM PHYSIOTHERAPY WA PTY LTD, Bentley, Western Australia (AU)

(72) Inventors: Jordan Timothy POWELL, Bentley, Western Australia (AU); Edward Joseph KHOURY, Bateman, Western Australia (AU)

(21) Appl. No.: 16/622,655

(22) PCT Filed: Jun. 13, 2018
(86) PCT No.: PCT/AU2018/050585

§ 371 Date: Dec. 13, 2019

(30) Foreign Application Priority Data
Jun. 14, 2017 (AU) 2017902263
Oct. 19, 2017 (AU) 2017904237

(51) Int. Cl. A61C 5/00; A61F 5/058

(52) U.S. Cl. CPC A61C 5/007; A61F 5/05891

(57) ABSTRACT

A jaw support is provided that includes a chest engaging member provided to rest on an upper area of the chest of a user below the jaw, a jaw support member connected to the chest engaging member and a neck support extending around a rear side of the neck of the user. The chest engaging member and the jaw support member comprise U-shaped first and second elongate members, respectively. The jaw support member is pivotable between a first position, in which it is adjacent the chest engaging portion, and a second position, in which it moves away from the chest engaging member to support the chin. The first and second elongate members each comprise flat elongate members having corresponding shapes.

### FIELD OF THE INVENTION

The present invention relates to a device for supporting the jaw.

### BACKGROUND TO THE INVENTION

When undergoing dental procedures, it is often necessary for the jaw to be held open for extended periods of time. During these periods, supporting the jaw in the open position can put significant strain on the muscles surrounding the jaw, thereby resulting in the possibility of injury.

Devices have been proposed which may be used to support the jaw under the chin, thereby alleviating muscle strain. Such devices however have been relatively cumbersome and difficult to use. The present invention relates to a device provided for supporting the jaw which is compact, comfortable, easy to use and relatively cost effective.

### SUMMARY OF THE INVENTION

According to one aspect of the present invention there is provided a jaw support comprising:

a chest engaging member provided to rest on an upper area of the chest of a user below the jaw, the chest engaging member comprising a U-shaped first elongate member;
a neck support extending from the chest engaging member around a rear side of the neck of the user; and
a jaw support member connected to the chest engaging member, the jaw support member comprising a U-shaped second elongate member;
wherein the jaw support member is pivotable between a first position, in which the jaw support member is adjacent the chest engaging portion, and a second position, in which the jaw support member moves away from the chest engaging member such that the user may rest their chin on the jaw support member, the first and second elongate members comprising flat elongate members having corresponding shapes such that, in the closed configuration, a surface of the second elongate member is adjacent and parallel to a surface of the first elongate member along the lengths thereof.

Preferably first and second ends of the second elongate member are rotatably mounted to respective first and second ends of the first elongate member such that the second elongate member is rotatable about an axis passing through the first and second ends of the first elongate member.

Preferably the first and second ends of the first and second elongate members lie in parallel planes and intermediate portions of the first and second elongate members are twisted relative to the first and second ends such that, in use, when the first and second ends are adjacent sides of the neck, the intermediate portions lie parallel to the surface of the chest of the user.

Preferably when the U-shaped first elongate member is oriented in a generally horizontal plane, the first and second ends of the first and second elongate members are oriented in vertical planes and the intermediate portions of the first and second elongate members are angled relative to a vertical plane such that the intermediate portions are at or near horizontal.

In a preferred embodiment, the intermediate portion of the jaw support member includes an indent to receive the chin of a user.

In a preferred embodiment, the neck support comprises first and second arms extending outwardly away from first and second ends of the first elongate member respectively.

Preferably the first and second arms comprise relatively flat curved members such that the first and second arms curve around towards the rear of the neck of the user.

Preferably the first elongate member and the first and second arms are integrally formed from a resilient flexible material.

In a preferred embodiment, distal ends of the first and second arms are separated by a gap.

Preferably a strap is provided to connect between distal ends of the first and second arms.

In a preferred embodiment, a locking mechanism is provided between the first and second ends of the jaw support member and the first and second ends of the chest engaging member such that the jaw support member may be retained in one or more positions relative to the chest engaging member.

In a preferred embodiment, the locking mechanism is moveable between a first position in which the jaw support mechanism is restricted from moving towards the chest engaging portion but may move away from the chest engaging portion and a second position, in which the jaw support member is moveable towards the chest engaging portion.

Preferably the locking mechanism is moveable from the first position to the second position by operation of actuators provided adjacent the connection of the first and second elongate members.

Preferably ends of the second elongate member include cylindrical end portions having openings for receiving protrusions on ends of the first elongate member and wherein the locking mechanism is provided within the openings.

Preferably the actuators comprise levers extending radially outwardly from the cylindrical end portions.

In a preferred embodiment, the locking mechanism comprises:

a part cylindrical protrusion extending outwardly from ends of the first elongate member defining an arcuate gap;
an inner cylindrical protrusion located inside the part cylindrical protrusion;
a ring including the lever, the ring being located about the part cylindrical protrusion having an arcuate protrusion located in the gap for rotational movement between a first and a second position;
an arcuate spring clip located between the part cylindrical protrusion and the inner cylindrical protrusion having an outer end located adjacent the gap;
wherein movement of the arcuate protrusion from the first position to the second position moves the outer end of the arcuate spring clip inwardly to disengaged from teeth provided around an inner surface of the cylindrical end portion, thereby allowing the jaw support member to rotate towards the chest engaging portion.

Preferably the engagement between the outer end and the teeth is such that rotation of the cylindrical end portion in a direction which would move the jaw support member away the frame is not restricted with the ring in the first position.

### BRIEF DESCRIPTION OF THE DRAWINGS

The invention will now be described, by way of example, with reference to the following drawings, in which:

FIG. 1 is an upper perspective view of a jaw support in accordance with the present invention in a closed configuration;

FIG. 2 is a side view of the jaw support of FIG. 1 in the closed configuration;

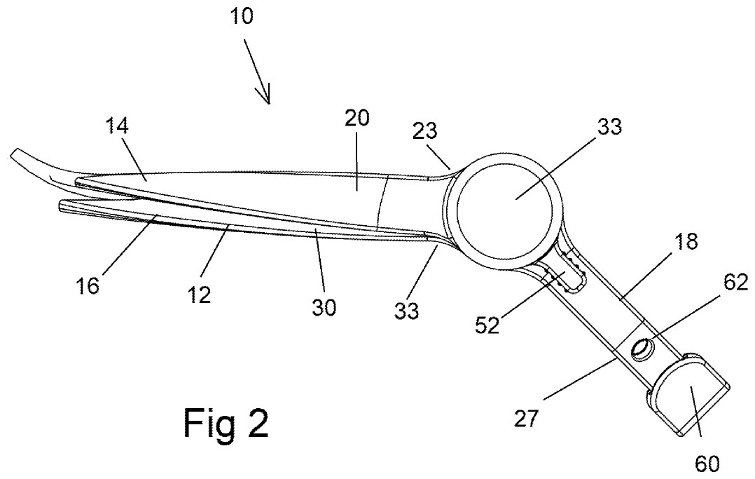

FIG. 3 is a rear view of the jaw support of FIG. 1 in the closed configuration;

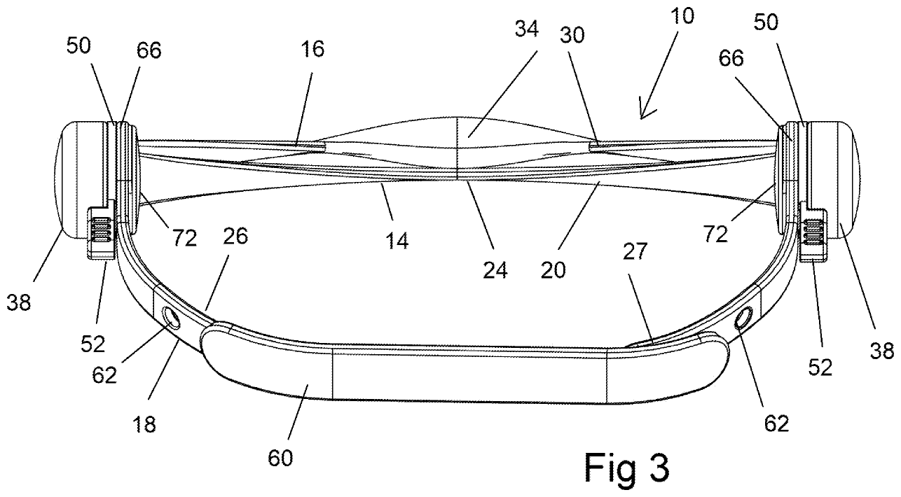

FIG. 4 is a front view of the jaw support of FIG. 1 in the closed configuration;

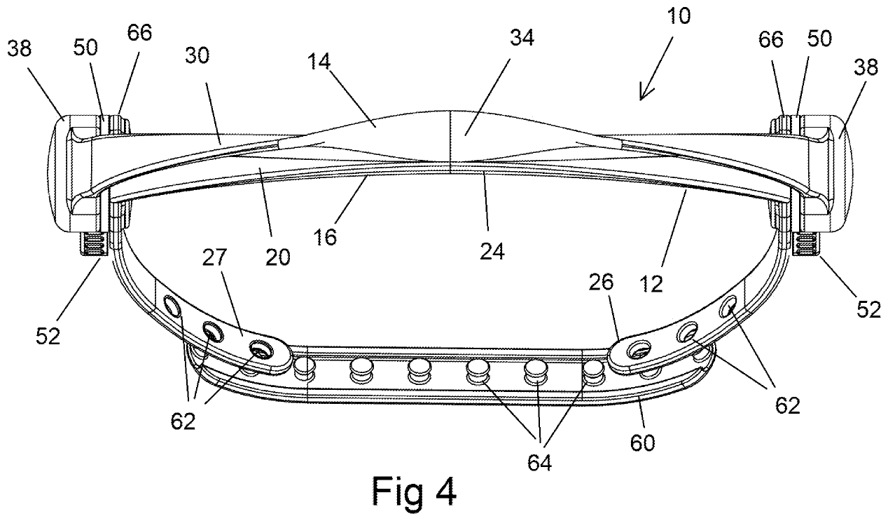

FIG. 5 is a top view of the jaw support of FIG. 1 in the closed configuration;

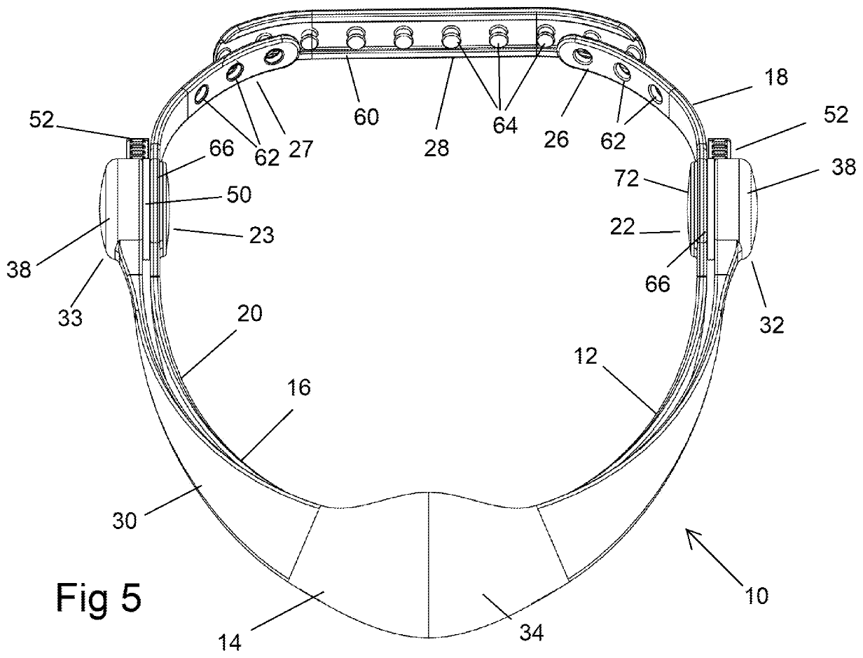

FIG. 6 is a bottom view of the jaw support of FIG. 1 in the closed configuration;

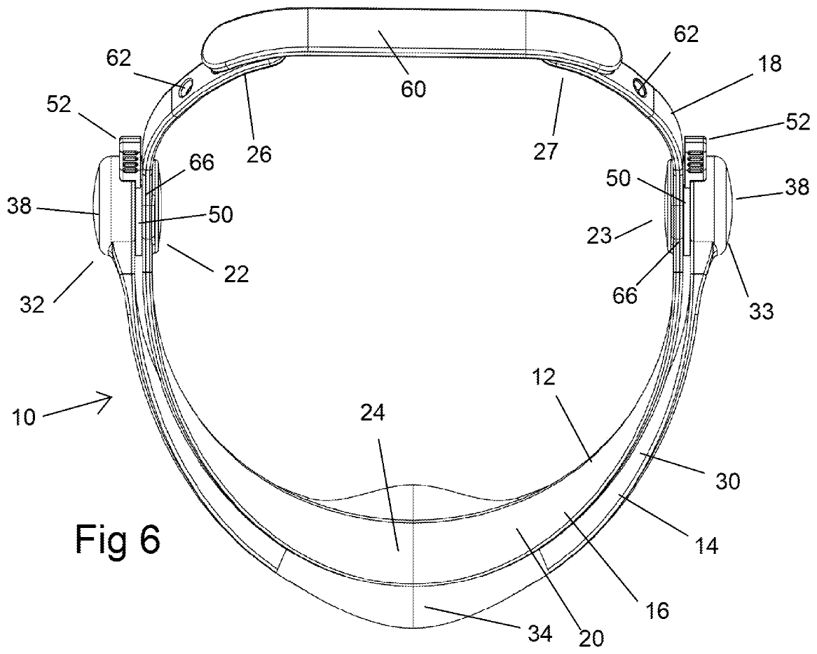

FIG. 7 is an exploded view of the jaw support of FIG. 1;

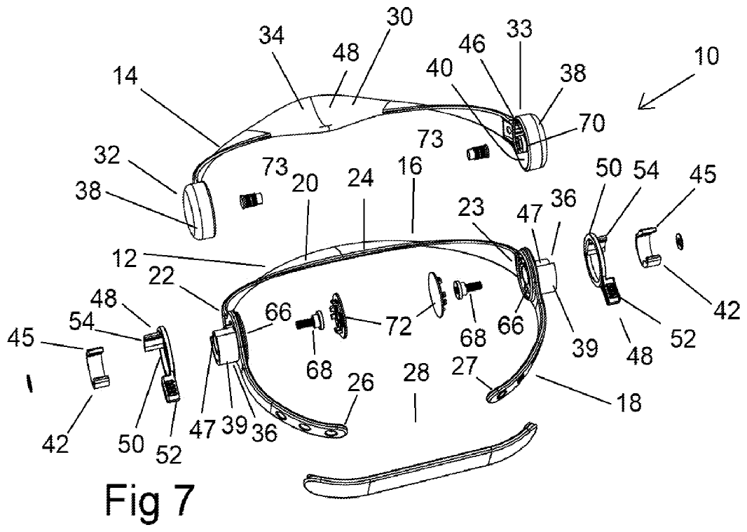

FIG. 8 is a top cross sectional view of the jaw support of FIG. 1;

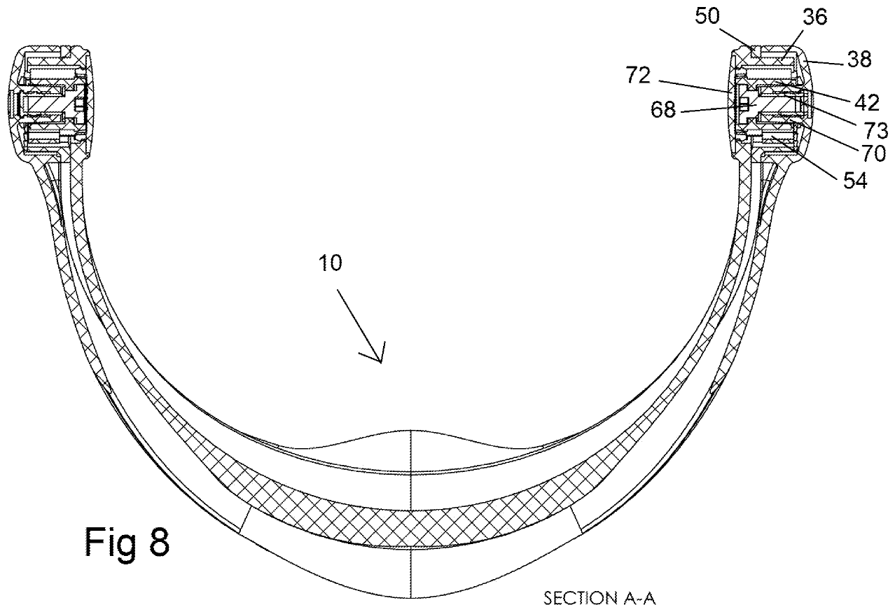

FIG. 9 is an exploded cross sectional view of the connection between the jaw support member and the chest engaging member;

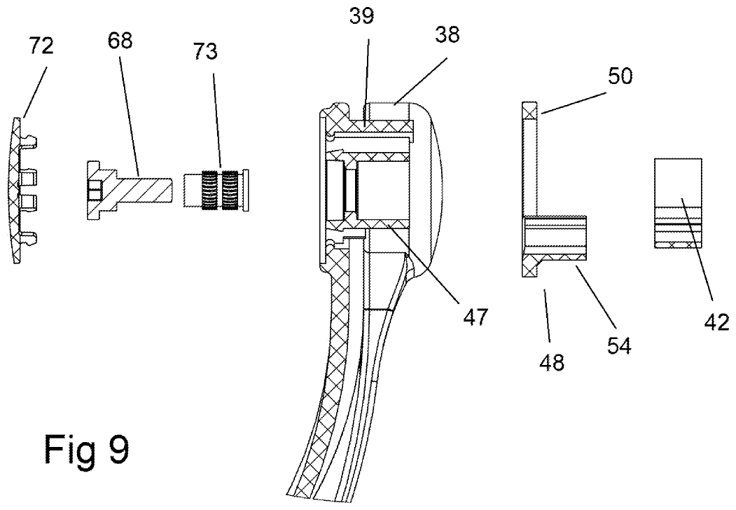

FIG. 10 is a side view of the jaw support member of FIG. 11 with the locking mechanism in a first position;

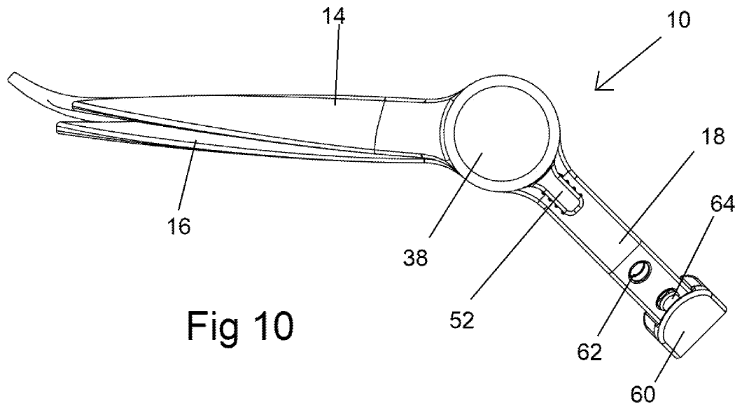

FIG. 11 is a side view of the jaw support member of FIG. 1 with the locking mechanism in a second position;

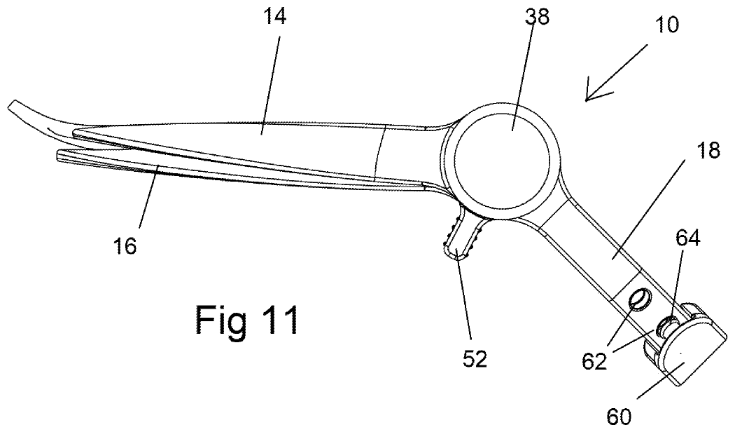

FIG. 12 is a view of the locking mechanism in the first position;

FIG. 13 is a view of the locking mechanism in the second position;

FIG. 14 is an upper perspective view showing movement of the jaw support member between the open and closed positions;

FIG. 15 is a side view showing movement of the jaw support member between the open and closed positions.

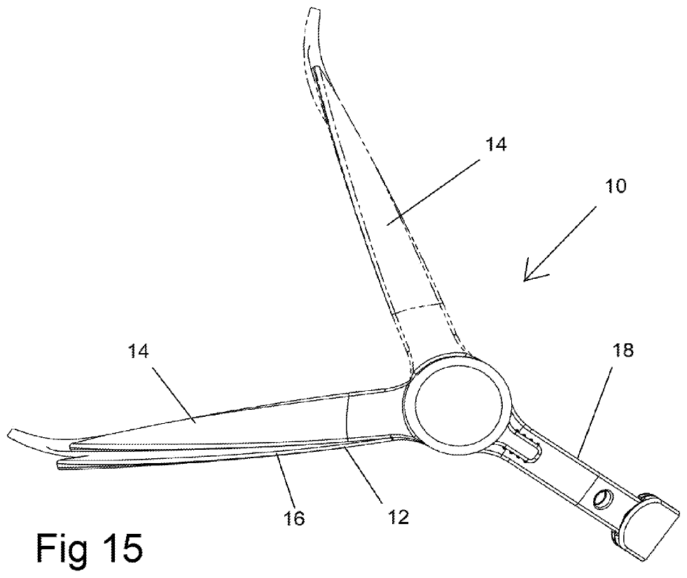

FIG. 16 is an upper perspective view of the jaw support of FIG. 1 in use;

FIG. 17 is a top view of the jaw support of FIG. 1 in use;

FIG. 18 is front view of the jaw support of FIG. 1 in use; and

FIG. 19 is a side view of the jaw support of FIG. 1 in use.

### DETAILED DESCRIPTION OF PREFERRED EMBODIMENTS

Referring to the Figures, there is shown a jaw support 10 comprising a frame 12 and a jaw support member 14. The frame 12 is provided to engage with the upper body of a user and comprises a chest engaging member 16 and a neck support 18.

The chest engaging member 16 is provided to engage with an upper portion of the chest of the user below the chin. The neck support 18 extends from the chest engaging member 16 and is provided to engage around a rear side of the neck of the user in order to hold the frame 12 in place in use.

The chest engaging member 16 comprises a curved first elongate member 20 being generally U-shaped. The chest engaging member 16 is curved such that when opposed first and second ends 22 and 23 are located on lateral opposed sides of the neck of the user, an intermediate portion 24 extends across an upper portion of the chest of the user such that the intermediate portion 24 rests generally above the sternum.

The first elongate member 20 comprises a flat elongate wall such that the thickness of the first elongate member 20 is relatively small in comparison to the width of the first elongate member 20. The first and second ends 22 and 23 of the first elongate member 20 lie in generally parallel planes. When the chest engaging member 16 is in place during use, the first and second ends 22 and 23 lie generally in parallel vertical planes either side of the neck.

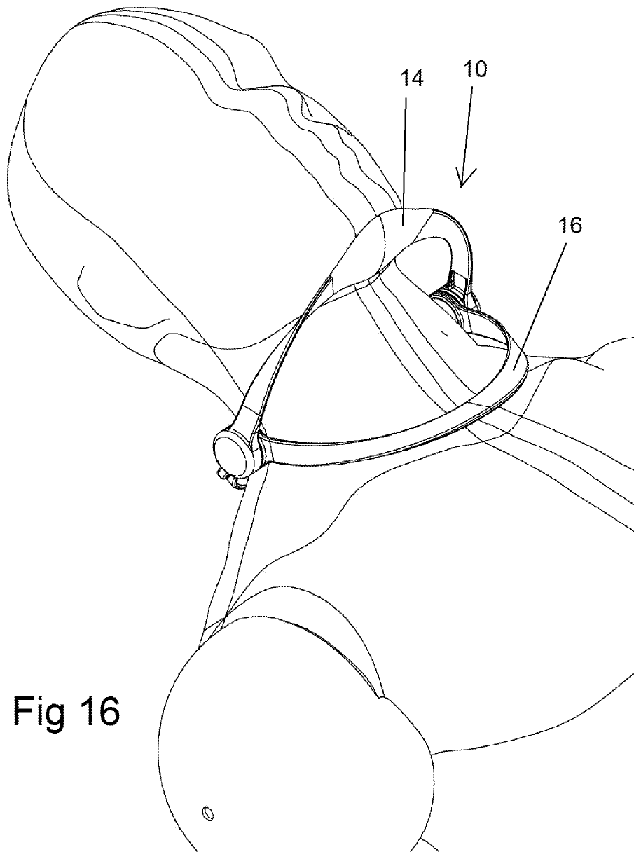

The intermediate portion 24 of the first elongate member 20 is twisted relative to the first and second ends 22 and 23 thereof such that, in use, the intermediate portion 24 lies generally parallel to the surface of the chest of the user, as can be seen in FIG. 16. That is, while the U-shaped first elongate member 20 is oriented in a generally horizontal plane, the first and second ends 22 and 23 of the first elongate member 20 are oriented in vertical planes and the intermediate portion 24 is angled relative to a vertical plane such that the intermediate portion 24 is at or near horizontal. With the first and second ends 22 and 23 of the first elongate member 20 located either side of the neck of the user, the intermediate portion 24 can therefore rest comfortably on the sternum of the user.

The neck support 18 in the embodiment shown comprises first and second arms 26 and 27. The first arm 26 extends outwardly away from the first end 22 of the first elongate member 20 and the second arm 27 extends outwardly away from the second end 23 of the first elongate member 20. The first and second arms 26 and 27 also comprise elongate walls such that the thickness of the first and second arms 26 and 27 is relatively small in comparison to the width of the first and second arms 26 and 27. The first and second arms 26 and 27 are curved such that the first and second arms 26 and 27 extend from ends of the first elongate member 20 around the rear of the neck of the user.

The first elongate member 20 and the first and second arms 26 and 27 are preferably integrally formed from a resilient flexible material, such as a plastic material. Distal ends of the first and second arms 26 and 27 are separated by a gap 28. The flexible resilient nature of the first elongate member 20 and the first and second arms 26 and 27 allows the gap 28 to be widened by pulling apart the first and second ends 22 and 23 of the frame 12. The frame 12 may then be placed around the neck for use.

The frame 12 is provided also with a connecting strap 60. The strap 60 extends from a distal end of the first arm 26 to a distal end of the second arm 27. The strap 60 is connectable to the first and second arms 26 and 27 to secure the jaw support 10 around the neck of the user. In the embodiment shown, the arms 26 and 27 include holes 62 along the length thereof and the strap 60 includes lugs 64 along the length thereof. The lugs 64 can be inserted into the holes 62 to adjust the distance between the arms 26 and 27.

The jaw support member 14 comprises a second elongate member 30 having a first end 32 pivotally secured to the first end 22 of the chest engaging member 16 and a second end 33 pivotally secured to the second end 23 of the chest engaging member 16. The second elongate member 30 also comprises a flat elongate wall such that the thickness of the second elongate member 30 is relatively small in comparison to the width of the second elongate member 30.

The first and second ends 32 and 33 lie in parallel planes either side of the neck in use. The first end 32 of the second elongate member 30 is therefore located outside and adjacent the first end 22 of the first elongate member 20. The second end 33 of the second elongate member 30 is located outside and parallel to the second end 23 of the first elongate member 20.

The contour of the second elongate member 30 corresponds to the contour of the first elongate member 20 such that the second elongate member 30 includes also a twisted intermediate portion 34. The second elongate member 30 is therefore locatable in a closed configuration in which the surface of the second elongate member 30 is adjacent and parallel to the surface of the first elongate member 20 along the length thereof.

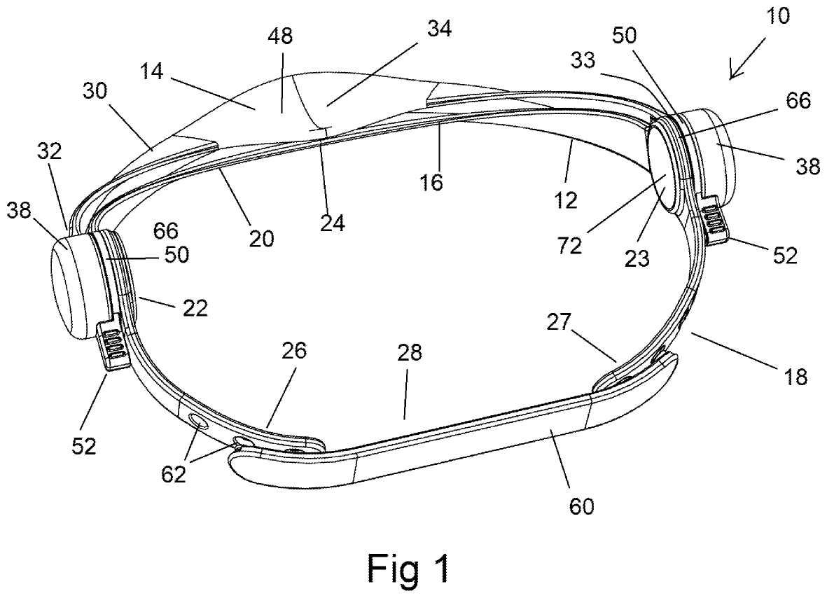

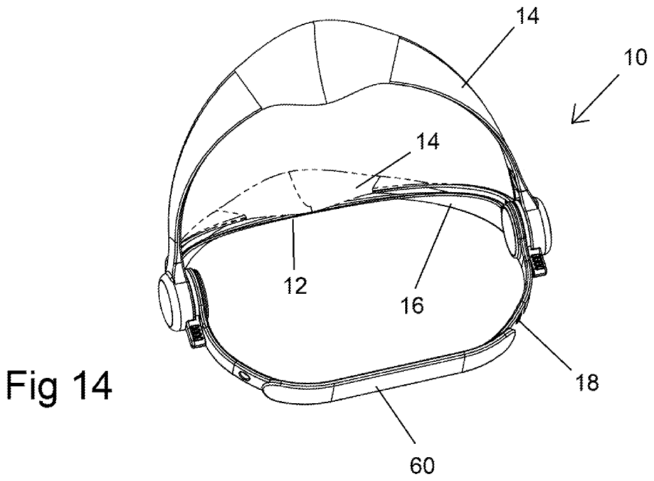

The first and second ends 32 and 33 of the jaw support member 13 are rotatably mounted to the outer surfaces of the first and second ends 22 and 23 of the chest engaging member 16 such that the jaw support member 14 can rotate from the closed configuration (as shown in FIG. 1) to an open configuration (as shown in FIG. 14). When rotating from the closed position to the open position, the intermediate portion 34 of the jaw support member 14 moves away from the intermediate portion 24 of the chest engaging member 16. The jaw support member 14 rotates about an axis passing between centres of the first and second ends 22 and 23 of the elongate member 20. As the jaw support member 14 moves to the open position, the intermediate portion 34 thereof therefore moves towards the chin of the user.

In the embodiment shown, first and second ends 22 and 23 of the chest engaging member 16 each include a circular end portion 66. The circular end portions 66 include protrusions 36 on outer surfaces thereof and the first and second ends 32 and 33 of the second elongate member 30 include cylindrical end portions 38 having closed outer ends such that the protrusions 36 are receivable in openings 40 defined by the cylindrical end portions 38 of the jaw support member 14. Securing bolts 68 pass through the circular end portions 66 into an inwardly extending shaft 70 provided within the openings 40 to connect together the first and second ends 22 and 23 of the chest engaging member 16 with the first and second ends 32 and 33 of the jaw support member 14 such that the jaw support member 14 and the chest engaging member 16 can rotate relative to each other. The shaft 70 may include an internal threaded metal insert 74 so that metal bolts 68 can engage with the shaft 70. Caps 72 are received over inner surfaces of the circular end portions 66 to cover ends of the bolts 68.

The jaw support 10 includes a locking mechanism provided to secure the jaw support member 14 relative to the chest engaging member 16 in a plurality of positions. In the embodiment shown, the locking mechanism prevents the jaw support member 14 from moving towards the chest engaging member 16 but allows movement of the jaw support member 14 away from the chest engaging member 16.

In the embodiment shown, the locking mechanism is provided within the openings 40 in the cylindrical end portions 38 and is operated by actuators comprising levers 52 extending radially outwardly from the cylindrical end portions 38. The levers 52 extend adjacent the first and second arms 26 and 27 and can be rotated about a central axis of the cylindrical end portions 38 between a first position, in which the jaw support member 14 is prevented from moving towards the chest engaging member 16, and a second position, in which the jaw support member 16 is free to move towards the chest engaging member 16.

The protrusion 36 in this embodiment includes a part cylindrical protrusion 39 defining an arcuate gap 37. An inner cylindrical protrusion 47 is provided located inside the part cylindrical protrusion 39. The actuator of the locking mechanism includes a ring 50 having the lever 52 extending therefrom. The ring 50 is located about the part cylindrical protrusion 39 and located in place when the protrusion 36 is received in the opening 40 in the cylindrical end portion 38 such that the ring 50 can rotate about the part cylindrical protrusion 39.

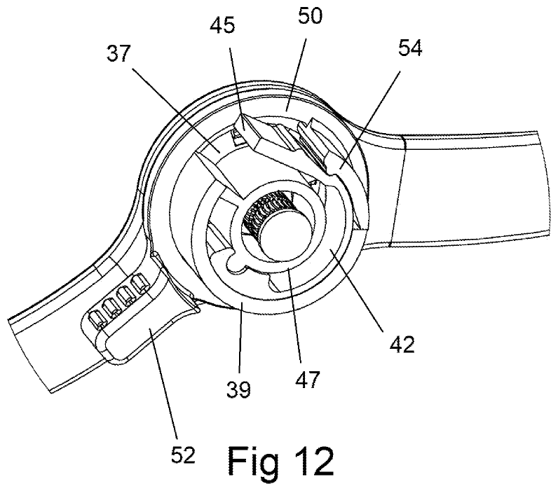

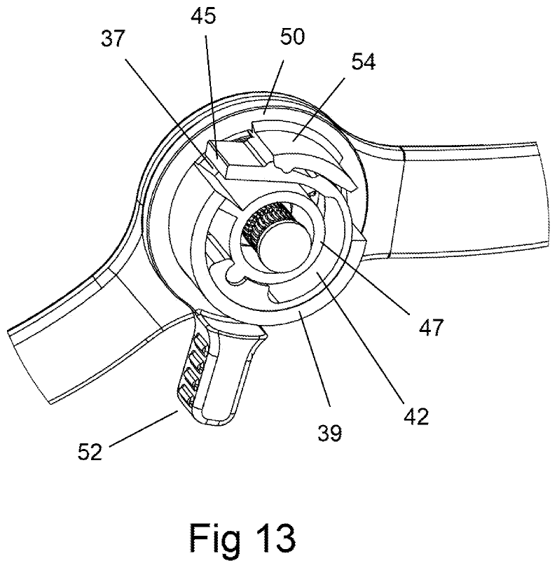

An arcuate protrusion 54 is provided on the ring 50 such that the arcuate protrusion 54 is received in the arcuate gap 37. The arcuate protrusion 54 is shorter than the arcuate length of the arcuate gap 37 such that the ring 50 can rotate between a first position (as shown in FIG. 12) and a second position (as shown in FIG. 13). An arcuate spring clip 42 is also provided which includes an outer end 45 provided to engage into teeth 46 provided around a portion of the inner surface of the cylindrical end portion 38. The arcuate spring clip 42 is provided between the part cylindrical protrusion 39 and the inner cylindrical protrusion such that the outer end 45 extends outwardly into the arcuate gap 37.

When the ring 50 is rotated to the first position, the arcuate protrusion disengages from the arcuate spring clip 42 such that the end 45 moves outwardly as shown in FIG. 12 through the arcuate gap 37. The end 45 is shaped to move into engagement with the teeth 46 such that rotation of the cylindrical end portion 38 in a direction which would move the jaw support member 14 towards the frame 12 is prevented. The flexibility of the arcuate spring clip 42 however lets the end 42 ride over the teeth 46 when the jaw support member 14 moves in the opposition direction. The ring 50 may be rotated by the lever 52 to the second position (as shown in FIG. 13) in which the arcuate protrusion 54 moves across the outer surface of the arcuate spring clip 42 to move the outer end 45 inwardly. The outer end 45 is thereby disengaged from the teeth 46 to allow the jaw support member 14 to move back to the closed position.

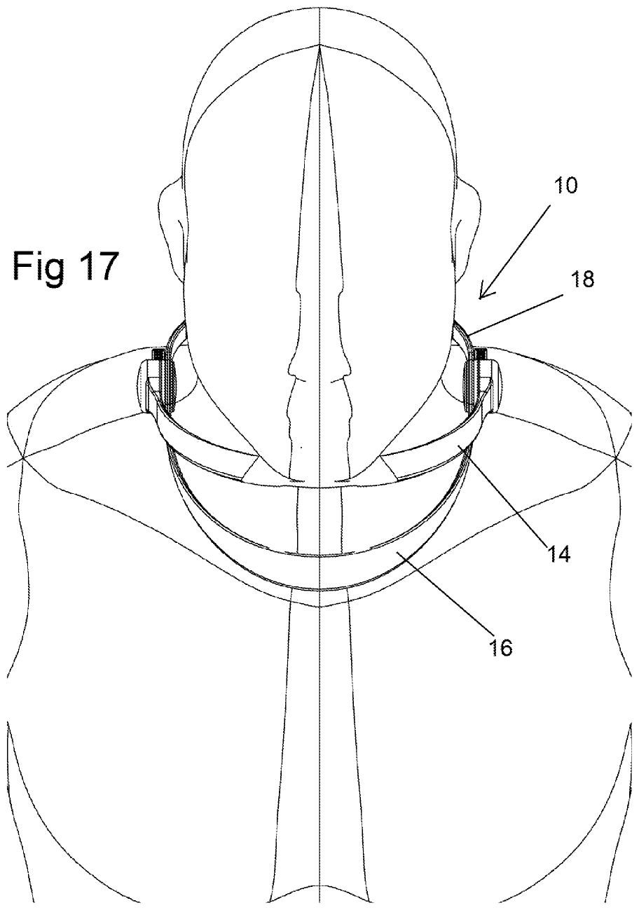

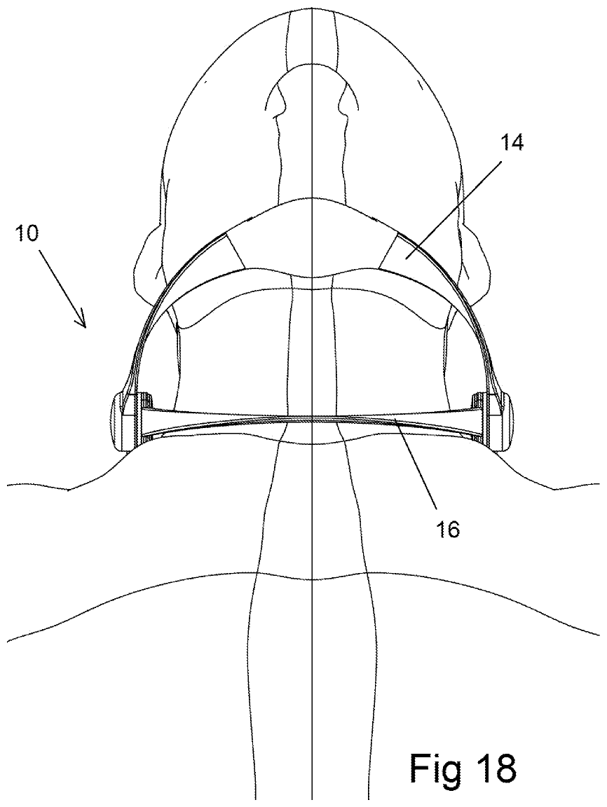

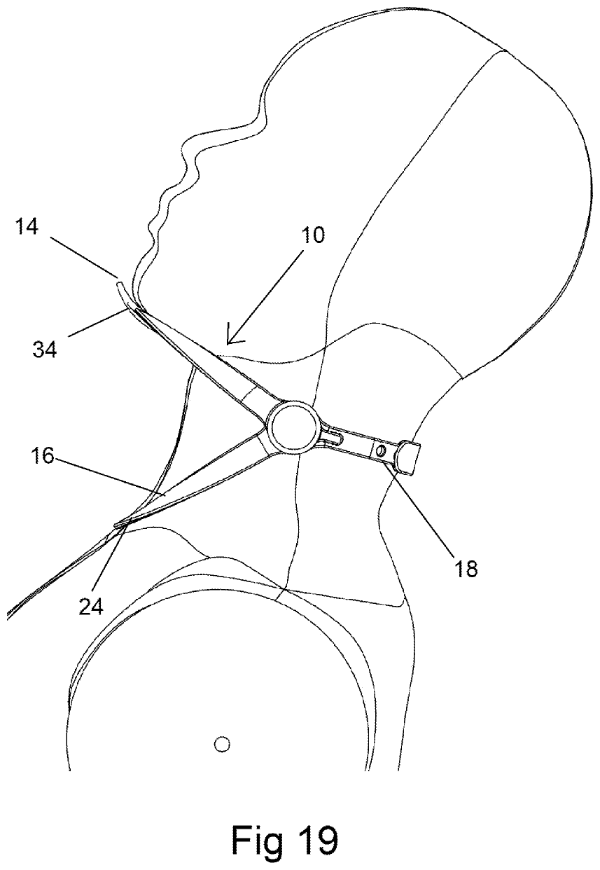

In use, the jaw support 10 may be stored in the closed configuration in which the jaw support member 14 and the chest engaging portion 16 are adjacent each other. When required for use, the frame 12 is placed around the neck of the user as shown in FIGS. 16 to 19. The jaw support member 14 is then moved to the open configuration to be placed against the chin of the user when the user's jaw is in the open position. The intermediate portion 34 of the jaw support member 14 includes an indent 48 to receive the chin of the user for additional comfort. The user can then rest their jaw against the jaw support member 14, the force of which is transferred by the chest engaging member 16 to the upper chest of the user. Stress is thereby taken away from the muscles around the jaw while the user is undergoing a procedure. When the jaw support 10 is no longer required, the lever 52 is moved to the second position to allow the jaw support member 14 to be moved back to the closed position adjacent the chest engaging member 16.

The jaw support 10 may be provided with a cover (not shown). The cover may comprise a disposable flexible plastic sheet formed into a shape such that it may be placed over the jaw support 10 in use. The plastic sheet may include elasticised edges such that it may be stretched over the jaw support 10 where it will engage with the jaw support 10 and remain in place. The jaw support 10 may therefore be used for multiple procedures by simply replacing the disposable cover.

It will be readily apparent to persons skilled in the relevant arts that various modifications and improvements may be made to the foregoing embodiments, in addition to those already described, without departing from the basic inventive concepts of the present invention.

1-17. (canceled)

18. A jaw support comprising:
a chest engaging member provided to rest on an upper area of the chest of a user below the jaw, the chest engaging member comprising a U-shaped first elongate member;
a neck support extending from the chest engaging member around a rear side of the neck of the user; and
a jaw support member connected to the chest engaging member, the jaw support member comprising a U-shaped second elongate member;
wherein the jaw support member is pivotable between a first position, in which the jaw support member is adjacent the chest engaging portion, and a second position, in which the jaw support member moves away from the chest engaging member such that the user may rest their chin on the jaw support member, the first and second elongate members comprising flat elongate members having corresponding shapes such that, in the closed configuration, a surface of the second elongate member is adjacent and parallel to a surface of the first elongate member along the lengths thereof.

19. The jaw support according to claim 18, wherein first and second ends of the second elongate member are rotatably mounted to respective first and second ends of the first elongate member such that the second elongate member is rotatable about an axis passing through the first and second ends of the first elongate member.

20. The jaw support according to claim 19, wherein the first and second ends of the first and second elongate members lie in parallel planes and intermediate portions of the first and second elongate members are twisted relative to the first and second ends such that, in use, when the first and second ends are adjacent sides of the neck, the intermediate portions lie parallel to the surface of the chest of the user.

21. The jaw support according to claim 20, wherein when the U-shaped first elongate member is oriented in a generally horizontal plane, the first and second ends of the first and second elongate members are oriented in vertical planes and the intermediate portions of the first and second elongate members are angled relative to a vertical plane such that the intermediate portions are at or near horizontal.

22. The jaw support according to claim 21, wherein the intermediate portion of the jaw support member includes an indent to receive the chin of a user.

23. The jaw support according to claim 18, wherein the neck support comprises first and second arms extending outwardly away from first and second ends of the first elongate member respectively.

24. The jaw support according to claim 23, wherein the first and second arms comprise relatively flat curved members such that the first and second arms curve around towards the rear of the neck of the user.

25. The jaw support according to claim 24, wherein the first elongate member and the first and second arms are integrally formed from a resilient flexible material.

26. The jaw support according to claim 25, wherein distal ends of the first and second arms are separated by a gap.

27. The jaw support according to claim 26, wherein a strap is provided to connect between distal ends of the first and second arms.

28. The jaw support according to claim 18, wherein a locking mechanism is provided between the first and second ends of the jaw support member and the first and second ends of the chest engaging member such that the jaw support member may be retained in one or more positions relative to the chest engaging member.

29. The jaw support according to claim 28, wherein the locking mechanism is moveable between a first position in which the jaw support mechanism is restricted from moving towards the chest engaging portion but may move away from the chest engaging portion and a second position, in which the jaw support member is moveable towards the chest engaging portion.

30. The jaw support according to claim 29, wherein the locking mechanism is moveable from the first position to the second position by operation of actuators provided adjacent the connection of the first and second elongate members.

31. The jaw support according to claim 30, wherein ends of the second elongate member include cylindrical end portions having openings for receiving protrusions on ends of the first elongate member and wherein the locking mechanism is provided within the openings.

32. The jaw support member in accordance claim 31, wherein the actuators comprise levers extending radially outwardly from the cylindrical end portions.

33. The jaw support according to claim 32, wherein the locking mechanism comprises:
a part cylindrical protrusion extending outwardly from ends of the first elongate member defining an arcuate gap;
an inner cylindrical protrusion located inside the part cylindrical protrusion;
a ring including the lever, the ring being located about the part cylindrical protrusion having an arcuate protrusion located in the gap for rotational movement between a first and a second position;
an arcuate spring clip located between the part cylindrical protrusion and the inner cylindrical protrusion having an outer end located adjacent the gap;
wherein movement of the arcuate protrusion from the first position to the second position moves the outer end of the arcuate spring clip inwardly to disengaged from teeth provided around an inner surface of the cylindrical end portion, thereby allowing the jaw support member to rotate towards the chest engaging portion.

34. The jaw support according to claim 33, wherein the engagement between the outer end and the teeth is such that rotation of the cylindrical end portion in a direction which would move the jaw support member away the frame is not restricted with the ring in the first position.
